(19) United States
(12) Patent Application Publication
(10) Pub. No.: US 2026/0041939 A1
(43) Pub. Date: Feb. 12, 2026

(54) METHODS FOR THE TREATMENT OF HYPERTENSION VIA TRANSCRANIAL-FOCUSED-ULTRASOUND

(71) Applicant: SUNNYBROOK RESEARCH INSTITUTE, Toronto (CA)

(72) Inventors: Kullervo HYNYNEN, Toronto (CA); Harriet LEA-BANKS, Toronto (CA)

(21) Appl. No.: 18/995,472

(22) PCT Filed: Jul. 21, 2023
(86) PCT No.: PCT/CA2023/050984

§ 371 Date: Jan. 16, 2025

Related U.S. Application Data
(60) Provisional application No. 63/391,201, filed on Jul. 21, 2022.

(51) Int. Cl. A61N 7/00; A61K 9/00; A61K 9/107; A61K 31/515; A61P 9/12

(52) U.S. Cl. CPC A61N 7/00; A61K 9/0019; A61K 9/1075; A61K 31/515; A61P 9/12; A61N 2007/0026

(57) ABSTRACT

A minimally invasive method for the reduction of hypertension is provided using focused ultrasound. After injection of ultrasound-responsive nanodroplets loaded with a therapeutic agent (e.g. barbiturates), focused ultrasound is delivered to a norepinephrine-producing region, such as the periaqueductal gray region, thereby locally releasing the therapeutic agent payload from the nanodroplets and achieving a therapeutic reduction in blood pressure. Acoustic emissions from vaporizing droplets may be employed, for example, to infer one or more therapeutic parameters based on a pre-established correlation. For example, plasma hormone content and/or the change in blood pressure may be inferred, thereby providing a means of real-time treatment monitoring. The present example methods may be employed to achieve a reduction in blood pressure for subjects exhibiting drug-resistant hypertension.

### CROSS-REFERENCE TO RELATED APPLICATION

This application claims priority to U.S. Provisional Patent Application No. 63/391,201, titled “METHODS FOR THE TREATMENT OF HYPERTENSION VIA TRANSCRANIAL-FOCUSED-ULTRASOUND” and filed on Jul. 21, 2022, the entire contents of which is incorporated herein by reference.

### BACKGROUND

The present disclosure relates to methods for neuromodulation that employ focused ultrasound.

Raised blood pressure, known as hypertension, effects a quarter of adults worldwide, and the prevalence in low-income countries continues to rise (WHO, 2022). Current strategies to reduce hypertension typically use pharmaceuticals.

Antihypertensive drugs have been in clinical use for over 6 decades, and are grouped into five major classes (Beta-blockers; diuretics; angiotensin-converting enzyme inhibitors; angiotensin II receptor blockers; calcium-channel blockers), which act on the central nervous system, reduce cardiac output, cause vasodilation, or alter enzyme secretion. However many of these drugs are ineffective or unsafe in drug-resistant hypertension and during pregnancy (Laurent, 2017).

Focused ultrasound (FUS), sound waves above 20 kHz with maximum intensity focused on a single target, can non-invasively modulate brain activity. This technique offers higher precision and deeper penetration than external electrical and magnetic fields, allowing specific anatomical brain regions to be targeted (Meng et al., 2021). The Food and Drug Administration (FDA) approved the use of focused ultrasound for the treatment of essential tremor in July 2016. By precise MRI-guided ablation of the thalamus—termed focused ultrasound thalamotomy—a significant reduction in hand tremor and enhanced motor function can be achieved (Elias et al., 2016) and has now been used to treat nearly 7,000 patients worldwide (InSightec). Since then, a similar procedure has been utilized in the treatment of Parkinson's Disease (Martínez-Fernandez et al., 2018), Obsessive Compulsive Disorder (OCD), and major depression disorder (Davidson et al., 2020), and confirmed to be safe in the treatment of epilepsy (Stern et al., 2021).

At lower intensities, non-ablative ultrasound exposures can transiently modulate neuronal activity (Fomenko et al., 2018). This has been shown preclinically in the treatment of epilepsy (Hakimova et al., 2015; Zhang, Li, Liu, et al., 2021; Zhang, Li, Lv, et al., 2021), and essential tremor (Sharabi et al., 2019). In a model of acute pain, sonication of the periaqueductal gray region (PAG) showed an analgesic effect (Zhang et al., 2022). In a rat model of hypertension, Li et al. showed that low-intensity ultrasound delivered daily to the ventrolateral periaqueductal gray (vIPAG) reduced systolic blood pressure, heart rate, and hypertension-related proteins in the plasma (Li et al., 2020). The study showed how ultrasound could achieve a similar reduction in blood pressure to DBS, non-invasively. However, the duration of reduced blood pressure following treatment was not reported. Therefore, it remains unclear whether 7 consecutive daily treatments were optimal or necessary, and whether the effects persisted to a clinically relevant time frame.

As well as direct neuromodulation, FUS is a non-invasive way to release anesthetic agents in the brain, using ultrasound-responsive drug carriers such as droplets for delivering propofol (Airan et al., 2017) and a barbiturate (Lea-Banks et al., 2020), and microbubbles for muscimol release (Ozdas et al., 2020). Combining FUS with locally released anesthetics allows the duration of neuromodulation to be controlled, extending the effect based on the kinetics of the drug. Previously, the present inventors have shown this offline neuromodulation using pentobarbital-loaded nanodroplets (PBND), showing persistent sensorimotor deficit in rats 60 min (Lea-Banks et al., 2020) and 90 min after the sonication had ended (Lea-Banks et al., 2021). Furthermore, encapsulation of anesthetic agents prevents off-target effects, such as general sedation, and increases the reliability of suppression in the focal region (Wang et al., 2018).

### SUMMARY

A minimally invasive method for the reduction of hypertension is provided using focused ultrasound. After injection of ultrasound-responsive nanodroplets loaded with a therapeutic agent (e.g. barbiturates), focused ultrasound is delivered to a norepinephrine-producing region, such as the periaqueductal gray region, thereby locally releasing the therapeutic agent payload from the nanodroplets and achieving a therapeutic reduction in blood pressure. Acoustic emissions from vaporizing droplets may be employed, for example, to infer one or more therapeutic parameters based on a pre-established correlation. For example, plasma hormone content and/or the change in blood pressure may be inferred, thereby providing a means of real-time treatment monitoring. The present example methods may be employed to achieve a reduction in blood pressure for subjects exhibiting drug-resistant hypertension.

Accordingly, in some aspects, there is provided a method of treating hypertension using focused ultrasound, the method comprising, after intravenous injection of nanodroplets loaded with a therapeutic agent:
delivering focused ultrasound to a norepinephrine producing region of the brain, wherein the focused ultrasound is configured to facilitate vaporization of the nanodroplets and release of the therapeutic agent.

In some example implementations of the method, the therapeutic agent comprises a barbiturate.

In some example implementations of the method, the therapeutic agent comprises a lipophilic anesthetic agent. The lipophilic anesthetic agent may have a molecular weight less than 400 Da with a logarithm of the partition coefficient approximately equal to 2.

In some example implementations of the method, the norepinephrine producing region comprises the periaqueductal gray region.

In some example implementations of the method, the norepinephrine producing region comprises the ventrolateral periaqueductal gray region.

In some example implementations of the method, the norepinephrine producing region comprises at least one of the locus coeruleus, thalamus, hypothalamus, neocortex and cerebellum.

In another aspect, there is provided a method of treating hypertension using focused ultrasound, the method comprising:
delivering focused ultrasound to a norepinephrine producing region of the brain, wherein the focused ultrasound is configured to induce repeated mechanical stress on the tissue by forming two or more closely spaced ultrasound pressure nodes where the neighboring nodes are in different acoustic phase thus creating a force between the nodes that stimulates or inhibits neuronal activity.

In some example implementations of the method, the ultrasound field is generated by one of a phased-array, lens, reflector, waveguide, and multiple overlapping ultrasound fields.

In some example implementations of the method, the norepinephrine producing region comprises the periaqueductal gray region.

In some example implementations of the method, the norepinephrine producing region comprises at least one of the locus coeruleus, thalamus, hypothalamus, neocortex and cerebellum.

In some example implementations of the method, the norepinephrine producing region comprises the ventrolateral periaqueductal gray region.

In some example implementations of the method, the focused ultrasound delivery is performed after intravenous injection of nanodroplets loaded with a therapeutic agent to facilitate vaporization of the nanodroplets and release of the therapeutic agent.

In some example implementations of the method, the focused ultrasound delivery is combined with a systemically delivered agent such as an anesthetic that will potentiate the ultrasound impact on brain tissue.

A further understanding of the functional and advantageous aspects of the disclosure can be realized by reference to the following detailed description and drawings.

### BRIEF DESCRIPTION OF THE DRAWINGS

Embodiments will now be described, by way of example only, with reference to the drawings, in which:

FIG. 1 is a flow chart illustrating an example method of hypertension reduction using delivery of transcranial focused ultrasound to the periaqueductal gray region (PAG) after injection of drug-loaded nano-droplets.

FIG. 2 illustrates an example transcranial focused ultrasound system.

FIG. 3 illustrates an example focused ultrasound headset.

FIG. 4 shows the persistence of blood pressure reduction in normotensive rats after a single sonication. (A) Schematic of non-invasive blood pressure measurement of awake rat in cylindrical rodent holder, with occlusion cuff and volume pressure recording (VPR) cuff, showing representative pressure measurement pre-treatment. (B) Schematic of focused ultrasound system targeting ventrolateral periaqueductal gray (vIPAG), (C) ultrasound pulsing scheme, (D) histological safety assessment showed no signs of hemorrhage or edema following sonication (scale bar 50 μm). (E) Systolic blood pressure, (F) diastolic blood pressure, and (G) heart rate before and after sonication. Lines show individual measurements from six healthy rats, significance is assessed from baseline (*p<0.05).

FIG. 5 shows the reduction in systolic and diastolic blood pressure of normotensive rats. Single treatment scheme, (A) treatment timeline, (B) heart rate, (C) systolic blood pressure (BP), (D) diastolic blood pressure (BP). Two consecutive treatments, (E) treatment timeline, (F) heart rate, (G) systolic blood pressure (BP), (H) diastolic blood pressure (BP). Error bars show one standard deviation, N=6, *p<0.05.

FIG. 6 shows the influence of sonicated brain region on reducing blood pressure in hypertensive rats. Change in systolic blood pressure, measured each day, from baseline, during (2 hr after treatment) to 6 days after treatment for (A) naïve, (B) PBND+FUS (active control), (C) FUS (active control), (H) PBND+FUS (vIPAG) and (I) FUS (vIPAG), shown for each rat. Noradrenaline plasma content 2 hr after sonication of (D-F) active control region and (J-L) vIPAG region plotted against acoustic emissions at the subharmonic frequency, systolic and diastolic blood pressure. Markers represent individual rats following treatment with FUS alone (circle) or pentobarbital-loaded nanodroplets (PBND) with FUS (triangle). Linear correlation is assessed with Pearson's coefficient (R²).

FIG. 7 shows the reduction of systolic and diastolic blood pressure in hypertensive rats. (A) Schematic of crossover study design, including five consecutive treatments. (B) Acoustic emissions from activated pentobarbital-loaded nanodroplets (PBND) map to the change in systolic blood pressure 2 hr after treatment. (C) Heart rate, (D) systolic blood pressure (BP) and (E) diastolic blood pressure (BP). Error bars show one standard deviation, N=4, *p<0.05.

FIG. 8 shows an immunohistochemistry analysis, demonstrating an increase in neuron activity following treatment. (A) Immunohistochemistry sections of the VLPAG stained for C-Fos (red), NeuN (green), and DAPI (blue), from 3 hypertensive rats (naïve, FUS only, PBND+FUS). Scale bar represents 100 μm. (B, C) C-Fos expression from the unsonicated (right) and sonicated (left) PAG region, shown in (D) schematic brain section. (E, F) NeuN positive cells in the unsonicated (right) and sonicated PAG region, and (G) proportion of C-Fos positive cells that were also NeuN positive, N=1.

FIG. 9 shows the habituation to blood pressure and heart rate measurements. (A1-A3) Normotensive rats and (B1-B3) SHRs were trained for 5 days prior to treatment to increase tolerance, reduce tail movement, and reduce measurement variation (standard deviation, STD) in blood pressure and heart rate (beats per minute, bpm).

FIG. 10 shows weight and temperature assessment for (A) healthy normotensive Wistars (n=6) and (B) spontaneously hypertensive rats (n=4) during habituation, treatment and post-treatment follow-up. Error bars represent one standard deviation, and no significance (NS) was found between groups (p>0.05).

FIG. 11 shows plasma hormone assessment of adrenaline levels in spontaneously hypertensive rats, showed no correlation with systolic or diastolic blood pressure (BP), assessed with Pearson's coefficient (R²<0.1).

FIG. 12 shows representative images of histological safety assessment performed using (A) TUNEL and (B) H&E staining to identify hemorrhage or cell apoptosis.

No damage was seen in the sonicated region of normotensive or hypertensive rats.

FIG. 13 is a table presenting the distribution of animals across experimental groups.

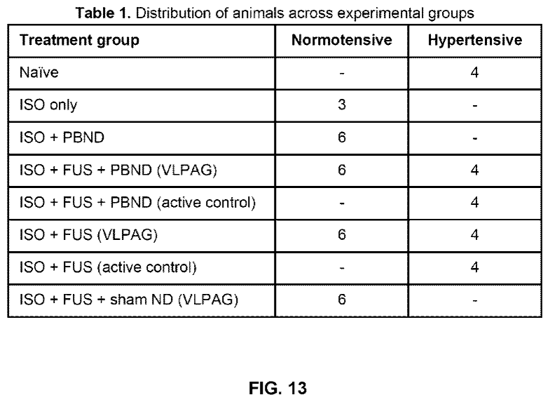

FIGS. 14A, 14B, 14C and 14D provide experimental results that demonstrate a dose-dependence of ultrasound-mediated reduction in blood pressure.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. Unless otherwise specified, the terms “about” and “approximately” mean plus or minus 25 percent or less.

It is to be understood that unless otherwise specified, any specified range or group is as a shorthand way of referring to each and every member of a range or group individually, as well as each and every possible sub-range or sub-group encompassed therein and similarly with respect to any sub-ranges or sub-groups therein. Unless otherwise specified, the present disclosure relates to and explicitly incorporates each and every specific member and combination of sub-ranges or sub-groups.

As used herein, the term “on the order of”, when used in conjunction with a quantity or parameter, refers to a range spanning approximately one tenth to ten times the stated quantity or parameter.

Unless defined otherwise, all technical and scientific terms used herein are intended to have the same meaning as commonly understood to one of ordinary skill in the art. Unless otherwise indicated, such as through context, as used herein, the following terms are intended to have the following meanings:

As noted above, although previous studies involving the delivery of focused ultrasound alone to the PAG have been successful in demonstrating a reduction of hypertension, however, the duration of such benefits was left unclear. As described in the examples provided below, the present inventors found that in the case of a hypertensive rat model, the delivery of focused ultrasound alone to the PAG resulted in only a subset of the treated rats exhibiting a reduction in blood pressure after focused ultrasound sonication. Moreover, in the hypertensive rats that did exhibit a reduction in blood pressure, the transient reduction persisted for only 24 hr after having delivered focused ultrasound to the PAG for five consecutive days.

The present inventors sought to improve the therapeutic effect and persistence of focused ultrasound sonication of the brain for an improved reduction in hypertension. Instead of merely sonicating the PAG region of the brain, the present inventors discovered that a combined therapeutic modality involving the use of focused ultrasound and barbiturate-loaded nanodroplets. It was discovered that when a subject is injected with barbiturate-loaded nanodroplets, the subsequent delivery of focused ultrasound to the PAG provided a dual-mechanism neuromodulation therapeutic benefit via the combined effect of the focused ultrasound on the PAG region and the impact of the local release within the PAG, via the focused ultrasound, of barbiturates from the nanodroplets.

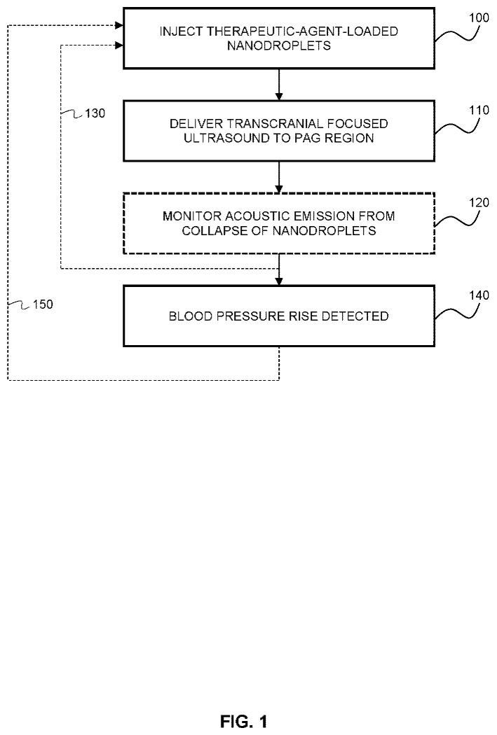

Accordingly, various example embodiments of the present disclosure employ focused ultrasound (FUS) in combination with barbiturate-loaded nanodroplets as a minimally invasive approach to reduce hypertension by modulating central brain activity. An example method of focused-ultrasound-based neuromodulation of hypertension via triggered release of barbiturate-loaded nanodroplets is illustrated in FIG. 1. At step 100, nanodroplets loaded with a therapeutic agent (e.g. pentobarbital) are injected into the bloodstream of a subject. Focused ultrasound is then delivered to the periaqueductal grey region (PAG) of the subject, as shown at step 110. The focused ultrasound is configured to facilitate the focused-ultrasound-triggered release of the therapeutic agent from the nanodroplets.

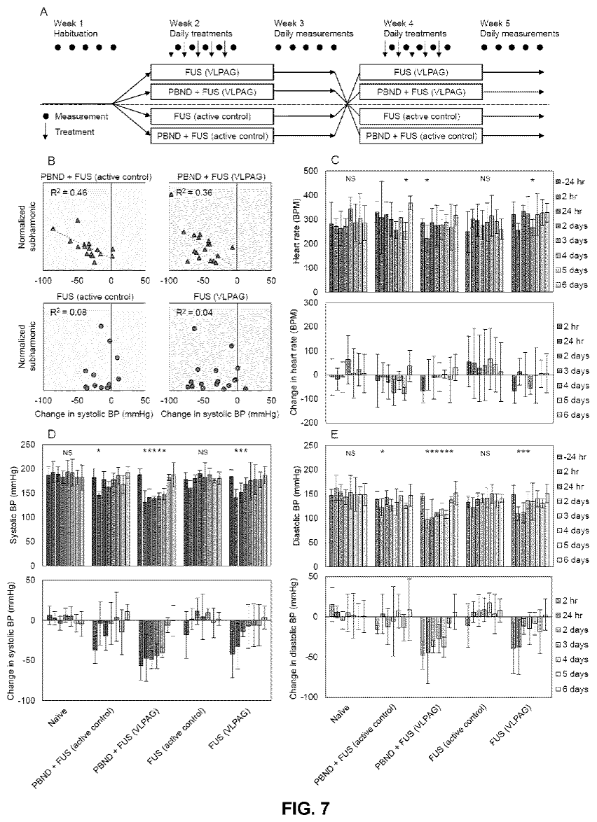

As shown at optional step 120, acoustic emission resulting from the collapse of the nanodroplets may be monitored, for example, using one or more of the transducers employed to deliver the focused ultrasound, and/or via one or more separate transducers. For example, the detected acoustic emission may be employed to provide a feedback mechanism to control the delivery of focused ultrasound, and/or as a means to verify or determine that an effective dose has been delivered via vaporization of the nanodroplets, and/or as a means to determine or infer a reduction in blood pressure caused by the treatment. For example, at a sufficient ultrasound pressure, known as the vaporization threshold, nanodroplets transition from liquid to gas state, releasing the therapeutic and emitting a unique acoustic signal. The subharmonic frequency (half the transmit frequency) can be used as a marker of drug release (Lea-Banks et al. 2020, 2021). As described below, acoustic emissions were found by the inventors to map to the reduction in blood pressure with weak correlation (R²=0.46 and 0.36, Pearson's coefficient) (see FIG. 7B), whereas acoustic emissions detected from FUS alone showed no correlation with change in blood pressure (R²=0.08 and 0.04, Pearson's coefficient).

Referring again to FIG. 1, steps 100 and 110 (and optional step 120) may be repeated one or more times, at a suitable time interval, in order to effect a sustained reduction in blood pressure, as shown at 130. For example, the treatment may be repeated on two or more consecutive days. In some example implementations, the treatment is repeated on 3, 4, 5, 6, 7, 8, 9 or 10 consecutive days.

As shown at step 140, after one or more treatments including steps 100 and 110, a rise in blood pressure may be detected after a time interval, such as, for example, 5, 6, 7, 8, 9 or 10 days. Steps 100-130 may then be repeated to obtain another reduction in blood pressure, as shown at 150.

Nanodroplet vaporization can be driven by changes in pressure and/or changes in temperature. The underlying mechanisms are termed mechanical or thermal (Lea-Banks et al. 2019, JCR). Mechanically-driven vaporization dominates when using low ultrasound frequencies (<2 MHz), typically used when sonicating through the skull. The vaporization threshold (the ultrasound pressure required to activate nanodroplets) is directly related to the frequency, meaning low frequency exposures (50 kHz-2 MHz) require low ultrasound pressures (0.2-3.0 MPa), with burst lengths between 1 ms-100 ms. The droplets can be vaporized also with shorter bursts but may require higher pressure amplitudes. In a rodent model, pentobarbital-loaded nanodroplets were found to remain acoustically active in circulation for up to 20 min following a single bolus injection (Lea-Banks et al. 2020, Theranostics). Therefore, sonication durations of 1-30 min following bolus administration may be used effectively, but the sonication may be extended if nanodroplets are delivered by infusion or multiple bolus injections.

The PAG is one component in a pathway controlling cardiovascular function. The hypothalamic arcuate nucleus (ARC), ventrolateral periaqueductal gray (vIPAG), and the rostral ventrolateral medulla (rVLM) are essential in governing sympathoexcitatory cardiovascular reflex responses. Without intending to be limited by theory, the present inventors believe that these alternate brain regions may be susceptible to ultrasound-triggered therapeutic agent (e.g. barbiturates) delivery and also influence blood pressure. Furthermore, the examples provided below indicate that changes in norepinephrine (noradrenalin) excretion and clearance are associated with hypotensive effects. Therefore, the present example embodiments may have use targeting other centers of norepinephrine excretion throughout the norepinephrine system, such as the locus coeruleus, thalamus, hypothalamus, neocortex and cerebellum.

The present example embodiments employ a therapeutic agent (drug) loading strategy that is applicable to a wide range of therapeutic agents, including small molecule lipophilic agents. In the example case of nanodroplets having a lipid shell, since the drug is incorporated into the lipid shell, and then released and delivered across the intact blood-brain barrier, the molecular weight of the drug should be less than 400 Da and the LogP (logarithm of the partition coefficient) should be near 2. Non-limiting examples of suitable barbiturates with a molecular weight less than 400 Da with a logarithm of the partition coefficient approximately equal to 2 include amobarbital, alphenal, butabarbital, butalbital, butethal, pentobarbital, phenobarbital, secobarbital, and thiopental. Since all barbiturates are agonists of GABAA receptors, it is expected that a similar inhibitory pathway would be triggered using an alternate barbiturate, leading to a reduction in blood pressure.

While various examples of the present disclosure relate to barbiturate-loaded nanodroplets, it will be understood that the nanodroplets may be loaded with other therapeutic agents, such as, but not limited to, small lipophilic anesthetic agents. Non-limiting examples of small lipophilic anesthetic agents include benzocaine, bupivacaine (Marcaine), ketamine, levobupivacaine, lidocaine, prilocaine, procaine, propofol, ropivacaine, tetracaine, which have similar inhibitory effects on neuronal activity.

In some example implementations, the nanodroplets can be formed with a core comprising low-boiling point perfluorocarbons, such as, but not limited to, Decafluorobutane and octafluoropropane, with a shell comprising one or more of phospholipids, proteins (e.g. albumin), and polymers (e.g. amphiphilic copolymers). Example methods of fabrication nanodroplets are disclosed in US Patent Application No. US20130336891, which is incorporated herein by reference in its entirety. Example methods of fabrication nanodroplets loaded with therapeutic agents are disclosed in US Patent Application No. US20200368352, which is incorporated herein by reference in its entirety. In some example implementations, the focused ultrasound that is delivered to the periaqueductal gray region induces repeated mechanical stress on the tissue by forming two or more closely spaced ultrasound pressure nodes (or focii), where the neighboring nodes are in different (in optimal configuration opposite) acoustic phase, thus creating a force between the nodes that stimulates or inhibits neuronal activity.

Such an example implementation may be performed after injection of therapeutic-agent-loaded nanodroplets, or in the absence of injection of therapeutic-agent-loaded nanodroplets.

The ultrasound field may be generated by one of a phased-array, lens, reflector, waveguide, and/or multiple overlapping ultrasound fields, and/or any other method allowing the control of the ultrasound field.

As demonstrated in the examples provided below, after a single 10 min sonication, blood pressure in healthy Wistar rats was reduced for 6 hrs. Following 5 consecutive daily treatments of focused ultrasound (FUS) with pentobarbital-loaded nanodroplets (PBND), diastolic blood pressure of hypertensive rats was lowered for up to 5 days, whilst sonication of the frontal cortex had no effect on blood pressure. vIPAG stimulation was confirmed with immunohistochemistry showing increased neuron activity, and with enzyme-linked immunosorbent assays (ELISA) showing a change in plasma hormone content.

The results presented herein demonstrate the use of focused ultrasound as a tool for minimally invasive treatment of hypertension with clinically relevant time scales. Hypertension is the most common disorder in pregnancy. However, many anti-hypertension drugs pose significant risks associated with restricted fetal growth, decreased uteroplacental blood flow, and fetal loss (Podymow and August, 2011). Mixed evidence on health risks has led to differing regulatory approval and clinical use across the world; for example, labetalol is the first-choice treatment for high blood pressure during pregnancy in the UK, whereas it is avoided in North America (US FDA pregnancy category C).

Furthermore, drug-resistant hypertension excludes all existing pharmaceutical strategies. DBS has been investigated as an alternative. In 2005, a 61-year-old male with drug-resistant hypertension was admitted to hospital with chronic neuropathic pain and received deep brain stimulation of the periaqueductal gray area (PAG) (Green et al., 2007). In this first-in-human study, electrical stimulation of the PAG reduced blood pressure from 157.4/87.6 mmHg to 132.4/79.2 mmHg, which had been unresponsive to antihypertensive drugs. However, blood pressure returned to baseline 20 s after the stimulation was turned off. Two further clinical studies investigated the relationship between blood pressure reduction and analgesia, and potential long-term hypotensive effects (Pereira et al., 2010; Patel et al., 2011). However, DBS is an invasive procedure, associated with potential infections and hemorrhage (Fenoy and Simpson, 2014). Therefore, a new strategy to treat hypertension is needed, that is non-invasive, long-lasting, and does not require antihypertensive drugs.

One long-term focused ultrasound treatment for hypertension is achieved with renal denervation. Following ultrasound ablation of renal nerves, blood pressure was reduced for at least 2 months in a recent multicentre clinical study (Azizi et al., 2021). Ultrasound renal denervation has been approved in Europe for treating hypertension. However, restrictions placed on renal anatomy (including main renal artery diameter and length must be within a specified range) limit accessibility of this treatment. In the 2021 study, 108 patients were excluded based on these anatomical criteria. Furthermore, 17% of patients experienced lasting pain (>2 days) following the procedure.

The present disclosure demonstrates that transcranial focused ultrasound in combination with barbiturate-loaded nanodroplets offers a non-invasive method to directly stimulate the PAG, and a minimally invasive method to trigger the delivery of an anesthetic agent. Both approaches have been shown to reduce hypertensive blood pressure (180/140 mmHg) into the healthy range (140/100 mmHg). The use of PBND to locally deliver an anesthetic was found to extend the effect of FUS from 2 days to 5 days following treatment.

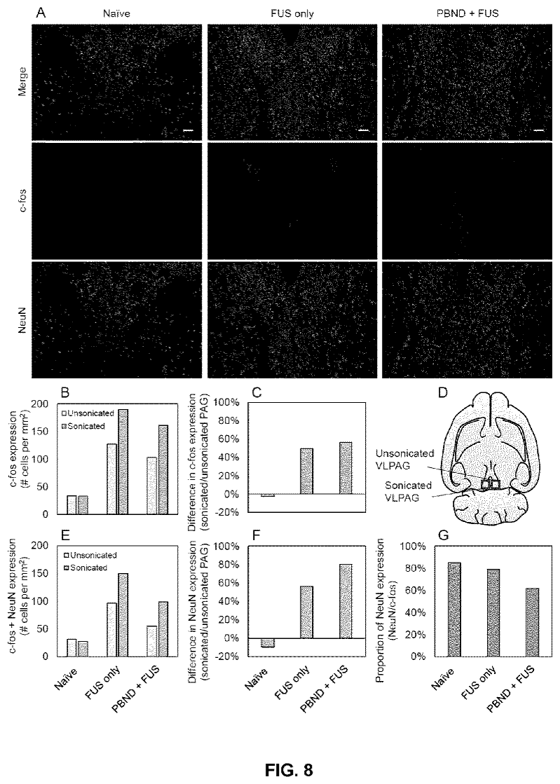

As shown in the examples below, with focused ultrasound alone, sonicating the frontal cortex had no effect on the blood pressure of hypertensive rats (FIG. 7). Comparing sonication of the active control region and vIPAG shows the reduction in blood pressure was dependent on brain anatomy. Decrease in blood pressure at 2 hr following PBND+FUS may be associated with non-specific drug release in active control region, upstream from the vIPAG and associated inhibitory pathways (FIGS. 8D, 8E). An acoustic controller to define the required pressure, combined with the local delivery of pentobarbital, increased the reliability of treatment. 4 of 4 hypertensive rats responded to PBND+FUS (vIPAG), whereas 1 of 4 rat treated with FUS alone did not respond.

Immunohistochemical analysis was also employed to probe the pathway for the hypotensive effect. C-Fos is an immediate early gene (IEG) associated with neuronal firing. In contrast to previous work of the present inventors delivering pentobarbital to the motor cortex where a suppression in C-Fos was mapped, in the present experiments, an increase in C-Fos expression is seen in the vIPAG region. Regional differences in C-Fos expression in response to pentobarbital have been investigated with microinjections of the anesthetic into specific anatomical regions of the brain. Pentobarbital, a GABAA receptor agonist, given through microinjection into the tuberomammillary nucleus of rats, has been found to increase c-FOS expression in the ventrolateral preoptic nucleus (VLPO), but suppress expression in the tuberomammillary nucleus, and leave expression in the locus coeruleus unchanged (Nelson et al., 2002). These anatomical differences are associated with the distribution of GABAergic-receptors, -neurons and -interneurons. Furthermore, by injecting GABA directly into the PAG region in rats, the significant portion of GABAergic receptors present in this region has been mapped, signaling the important GABAergic network responsible of far-reaching inhibitory pathways (Behbehani et al., 1990).

In the present example method, plasma hormone assays were performed to investigate the mechanisms driving blood pressure reduction. 2 hr following sonication of the vIPAG, a weak negative correlation was found between noradrenaline content in venous plasma samples and blood pressure, but no significant difference was found for adrenaline. Noradrenaline release is typically associated with an increase in metabolic function, heart rate and blood pressure. However, the relationship between GABAergic receptors in the PAG region and norepinephrine has been explored in the context of suppressing pain. Norepinephrine injections have been shown to inhibit pain-modulatory neurons (Basbaum and Fields, 1984), and an increase in noradrenaline release as a result of PAG stimulation has been reported in DBS, shown to inhibit dorsal horn neurons causing analgesia (Cui et al., 1999).

A further consideration is found in noradrenaline clearance, where increased circulating noradrenaline in venous plasma may signal a reduction in clearance rate (Esler et al., 1984). Noradrenaline clearance has been found to be slower in normotensive humans compared to those with hypertension (Grimm et al., 1980). Therefore, an increase in circulating noradrenaline may be a combined result of increasing noradrenaline release through PAG stimulation and lowered clearance rate. Without intending to be limited by theory, the present inventors hypothesize that the increase in PAG C-Fos expression and increase in plasma noradrenaline levels indicate PAG stimulation responsible for an inhibitory response, triggered by GABAergic networks, and resulting in reduced blood pressure.

The methods, examples and results disclosed herein demonstrate that FUS-mediated neuromodulation treatment for hypertension reduction is promising and can have clinical impact. Hypertension remains a fatal condition and prevalence continues to grow in low-income countries, but FUS offers a minimally invasive approach that has the potential to be optimized as a cost-effect long-lasting treatment for hypertension. Indeed, in the examples provided below, it is shown that that consecutive sonications in combination with anesthetic-loaded nanodroplets reduce hypertensive blood pressure into the heathy range for up to 5 days.

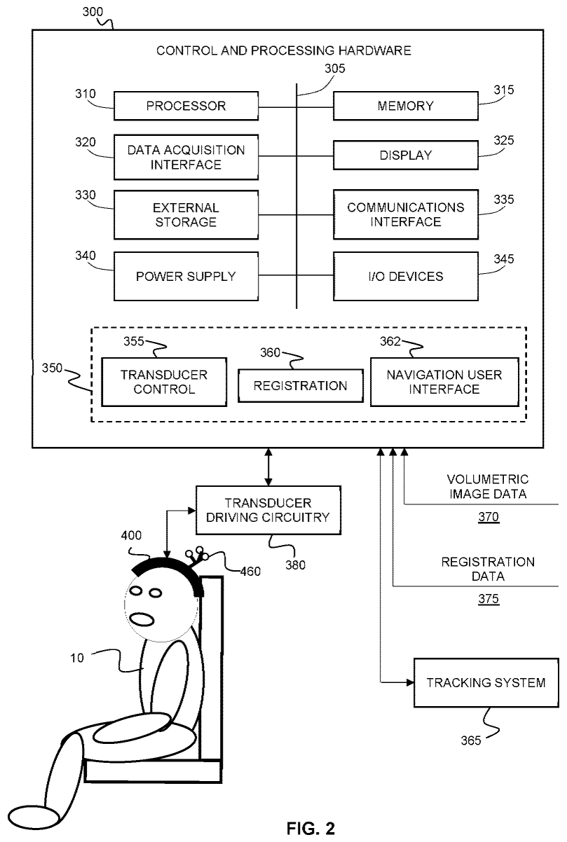

FIG. 2 provides a block diagram illustrating an example implementation of a system for performing diagnostic or therapeutic transcranial procedures. Control and processing hardware 300 is operably connected to the transcranial headset 100, optionally via transducer driver electronics/circuitry 380.

The control and processing hardware 300, which includes one or more processors 310 (for example, a CPU/microprocessor), bus 305, memory 315, which may include random access memory (RAM) and/or read only memory (ROM), a data acquisition interface 320, a display 325, external storage 330, one more communications interfaces 335, a power supply 340, and one or more input/output devices and/or interfaces 345 (e.g. a speaker, a user input device, such as a keyboard, a keypad, a mouse, a position tracked stylus, a position tracked probe, a foot switch, and/or a microphone for capturing speech commands).

Volumetric image data 370 and transducer registration data 375 may be stored on an external database or stored in memory 315 or storage 330 of control and processing hardware 300.

The tracking system 365 may optionally be employed to track the position and orientation of the patient, via detection of one or more fiducial markers 460 attached to the transcranial headset 400, and optionally one or more medical instruments or devices also having fiducial markers attached thereto. For example, passive or active signals emitted from the fiducial markers may be detected by a stereographic tracking system employing two tracking cameras. The transducer driving electronics/circuitry 380 may include, for example, but is not limited to, Tx/Rx switches, transmit and/or receive beamformers.

The control and processing hardware 300 may be programmed with programs, subroutines, applications or modules 350, which include executable instructions, which when executed by the one or more processors 310, causes the system to perform one or more methods described in the present disclosure. Such instructions may be stored, for example, in memory 315 and/or other storage.

In the example embodiment shown, the transducer control module 355 includes executable instructions for controlling the transducers of the transcranial headset 400 to deliver energy to the PAG region, based on the registration of the transducer positions and orientations with the volumetric image data as per the transducer registration data 375. For example, the transcranial headset 400 may support a plurality of phased-array transducers, and transducer control module 355 may control the beamforming applied (on transmit and/or receive) to deliver, based on the known positions and orientations of the phased array transducers relative to the volumetric image data, one or more focused energy beams to a region of interest in the far field regions of the transcranial ultrasound transducer array elements. The region of interest may be specified intraoperatively by a user (e.g. via a user interface controlled by control and processing hardware 300) or according to a pre-established surgical plan.

The registration module 360 may optionally be employed for registering volumetric image data 370 to an intraoperative reference frame associated with tracking system 365. The optional guidance user interface module 362 includes executable instructions for displaying a user interface showing spatially registered volumetric images for image-guided procedures. The registration module 360 may also intraoperatively receive spatial correction information based on a detected spatial offset between the transcranial frame and the patient's head (which may be provided by a subset of distance-sensing transducers) and employ this spatial correction information to dynamically adjust (e.g. correct) the registration between the transducers and the volumetric image data.

Although only one of each component is illustrated in FIG. 2, any number of each component can be included in the control and processing hardware 300. For example, a computer typically contains a number of different data storage media. Furthermore, although bus 305 is depicted as a single connection between all of the components, it will be appreciated that the bus 305 may represent one or more circuits, devices or communication channels which link two or more of the components. For example, in personal computers, bus 305 often includes or is a motherboard. Control and processing hardware 300 may include many more or less components than those shown.

The control and processing hardware 300 may be implemented as one or more physical devices that are coupled to processor 310 through one of more communications channels or interfaces. For example, control and processing hardware 300 can be implemented using application specific integrated circuits (ASICs). Alternatively, control and processing hardware 300 can be implemented as a combination of hardware and software, where the software is loaded into the processor from the memory or over a network connection.

Some aspects of the present disclosure can be embodied, at least in part, in software, which, when executed on a computing system, transforms a computing system into a specialty-purpose computing system that is capable of performing the methods disclosed herein. That is, the techniques can be carried out in a computer system or other data processing system in response to its processor, such as a microprocessor, executing sequences of instructions contained in a memory, such as ROM, volatile RAM, non-volatile memory, cache, magnetic and optical disks, or a remote storage device. Further, the instructions can be downloaded into a computing device over a data network in a form of compiled and linked version. Alternatively, the logic to perform the processes as discussed above could be implemented in additional computer and/or machine readable media, such as discrete hardware components as large-scale integrated circuits (LSI's), application-specific integrated circuits (ASIC's), or firmware such as electrically erasable programmable read-only memory (EEPROM's) and field-programmable gate arrays (FPGAs).

A computer readable medium can be used to store software and data which when executed by a data processing system causes the system to perform various methods. The executable software and data can be stored in various places including for example ROM, volatile RAM, non-volatile memory and/or cache. Portions of this software and/or data can be stored in any one of these storage devices. In general, a machine readable medium includes any mechanism that provides (i.e., stores and/or transmits) information in a form accessible by a machine (e.g., a computer, network device, personal digital assistant, manufacturing tool, any device with a set of one or more processors, etc.).

Examples of computer-readable media include but are not limited to recordable and non-recordable type media such as volatile and non-volatile memory devices, read only memory (ROM), random access memory (RAM), flash memory devices, floppy and other removable disks, magnetic disk storage media, optical storage media (e.g., compact discs (CDs), digital versatile disks (DVDs), etc.), among others. The instructions can be embodied in digital and analog communication links for electrical, optical, acoustical or other forms of propagated signals, such as carrier waves, infrared signals, digital signals, and the like. As used herein, the phrases “computer readable material” and “computer readable storage medium” refer to all computer-readable media, except for a transitory propagating signal per se.

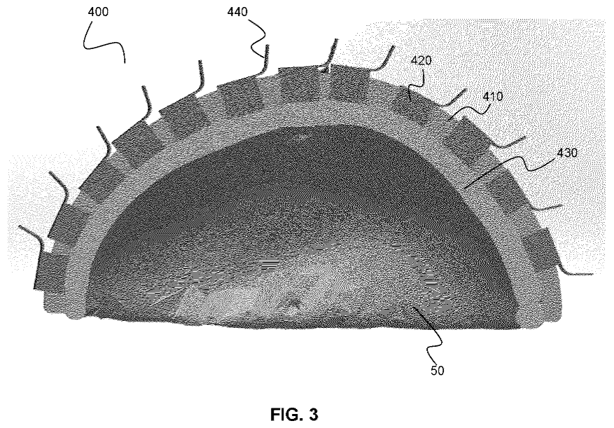

Referring now to FIG. 3, a non-limiting and example patient-specific headset 400 for performing transcranial diagnostic or therapeutic procedures is shown worn on the head 50 of a patient. The patient-specific headset 400, which includes a patient-specific frame (support structure) 410 that supports a plurality of transducers 420, conforms to the anatomical contour of at least a portion of the patient's head. The patient-specific frame 410, which is shown in cross-section in FIG. 3, mechanically supports transducers 420 in pre-selected positions and orientations. The transducers 420 may be used to transmit and/or receive energy for brain diagnostic or therapeutic purposes or for localization of the skull surface.

The patient-specific frame 410 includes a plurality of attachment interfaces for receiving and supporting the transducers 420. In the example embodiment shown in FIG. 3, the attachment interfaces are provided as apertures (recesses) into which the transducers 420 are placed. The transducers 420 may be affixed to the patient-specific frame 410 according to a wide variety of different means, such as, but not limited to, with an attachment mechanism (e.g. via fasteners that extend into the patient-specific frame 410, optionally into pre-formed holes), or an adhesive such as a glue. In the example implementation shown in FIG. 3, the transducers 420 are remotely interfaced with electronics through wires or through a flexible printed circuit board 440. The transducers 420 may be removably attachable to the patient-specific frame 410.

The example patient-specific headset shown in FIG. 3 may also include a coupling layer 430 that is provided adjacent to an inner surface of the patient-specific frame. The outer surface of the coupling layer 430 contacts distal surfaces of the transducers 420, and the inner surface of the coupling layer contacts the patient's head 50, thereby facilitating coupling of energy between the transducers in the patient-specific frame and the patient's head. The inclusion of the coupling layer 430, and the composition and/or geometry of the coupling layer, may be dependent on the type of transducers 420. For example, if the transducers 420 are ultrasound transducers, the coupling layer 430 may be an acoustic coupling layer that facilitates propagation of acoustic waves and reduces reflections at interfaces. In one example implementation, the coupling layer 430 includes an elastic membrane that retains a liquid layer between the transducer surfaces and the elastic membrane, such that coupling to the skin is achieved.

### EXAMPLES

The following examples are presented to enable those skilled in the art to understand and to practice embodiments of the present disclosure. They should not be considered as a limitation on the scope of the disclosure, but merely as being illustrative and representative thereof.

In the examples described below, two experiments were conducted: the first used healthy normotensive Wistar rats to assess the persistence of blood pressure reduction following a single 10 min sonication of the vIPAG region. A comparison was then made with effects of general anesthetic, FUS alone, FUS with nanodroplets, and FUS with pentobarbital-loaded nanodroplets (PBND) targeted to the vIPAG, given in a single treatment, or in a pair of treatments on consecutive days. The second experiment used a model of hypertension—spontaneously hypertensive rats (SHR)—to compare FUS alone and FUS with PBND, targeted to the vIPAG or the frontal cortex (as an active control), given in 5 treatments on consecutive days. Non-invasive blood pressure measurements were implemented in awake rats, plasma hormone assessment, and immunohistochemistry of neuron activity to probe the underlying mechanisms.

### Example 1: Methods

### Barbiturate-Loaded Nanodroplets

Ultrasound-responsive nanodroplets, 200 nm in diameter, were synthesized as previously described (Lea-Banks et al., 2021), following a modified condensation protocol (Sheeran et al., 2011, 2017; Matsunaga et al., 2012). Definity lipid solution (Lantheus Medical Imaging, USA) was combined with pentobarbital (Sigma Aldrich Millipore Sigma, USA) through tip sonication, degassed, and filled with decafluorobutane (C4F10). Precursor microbubbles were formed by agitation (VialMix, Lantheus Medical Imaging, USA), condensed at −10° C., washed by centrifugation (300 G, 8 min, 4° C.), and filtered at 0.8 μm (Minisart Syringe Filter, Sartorius, Germany). Nanodroplets were stored on ice before use and used within 2 hr of fabrication. Sham nanodroplets (sham ND) were fabricated in the same way, without the additional of pentobarbital.

### Animals

Two cohorts of rats were used to assess ultrasound-induced changes in blood pressure. Normotensive healthy Wistar rats (23 male, 4 female) were purchased from Charles River Laboratories (Wilmington, MA, USA), and were 319 +/−12 g (male) and 218 +/−3 g (female) on the first treatment day. Spontaneously hypertensive rats (SHR) (5 male, 5 female) were purchased from Envigo (Indianapolis, IN, USA), and were 256 +/−12 g (male) and 172 +/−8 g (female) on the first treatment day. Animals were housed in the Sunnybrook Research Institute animal facility (Toronto, ON, Canada) on a reverse light cycle and had access to food and water ad libitum. All animal procedures were approved by the Animal Care Committee at Sunnybrook Research Institute and are in accordance with the Canadian Council on Animal Care and ARRIVE guidelines.

### Animal Preparation

5% isoflurane (ISO) in medical air was used to induce general anesthesia, which was reduced to 2% for the duration of the ultrasound procedure. To allow coupling between the transducer and the scalp, hair was removed with an electric razor and depilatory cream, and a thin layer of ultrasound gel was applied to the scalp (Wavelength CL, ON, Canada). Ultrasound gel was spun down in a centrifuge tube before use to remove bubbles (1000 G, 10 min). A 22-gauge tail vein catheter was inserted. 2 mL of saline was given subcutaneously following blood collection, otherwise all injections were administered intravenously. The rat was positioned in the stereotactic frame of the FUS system, breathing into a nose cone connected to the ISO machine, and scavenged with an F/AIR charcoal filter. Warm saline bags were placed on the animal to maintain body temperature. PBND or sham ND were administered as a slow bolus through the tail vein catheter at a dose of 1.0 mL/kg (containing approximately 25 μg/mL of pentobarbital).

### Transcranial Fus System

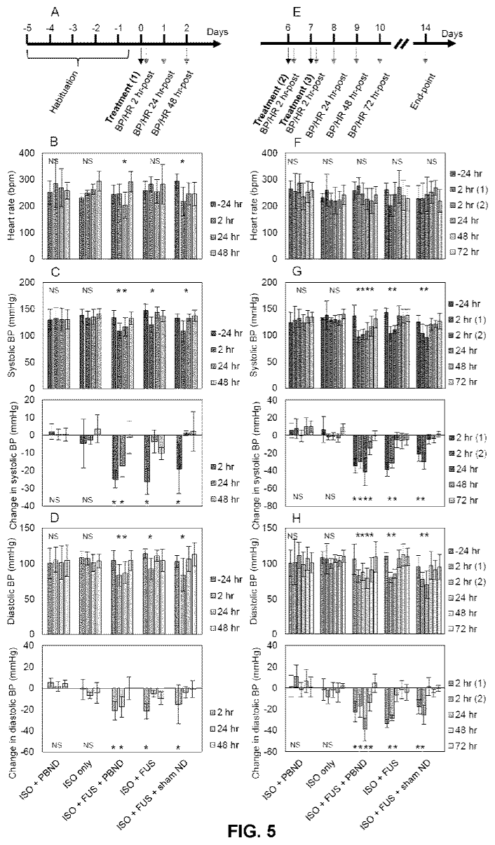

Focused ultrasound (FUS) at 540 kHz was targeted and delivered to the ventrolateral periaqueductal gray (vIPAG) region (D/V 5.8 mm, A/P −7.6 mm, L/M −0.6 mm relative to bregma) or the frontal cortex (D/V 4.0 mm, A/P 3.0 mm, L/M −0.6 mm relative to bregma) using a pre-clinical prototype system (an early prototype for the RK50, FUS Instruments Inc., ON, Canada) with atlas-based targeting. The geometry of the skull is co-registered with the focus of a single element transducer (centre frequency 540 kHz, element diameter 35 mm, focal length 25 mm), which has a central circular cut-out to house a narrowband PZT (lead zirconate titanate) hydrophone (centre frequency 270 kHz, element diameter 5 mm, unfocused) (FIG. 5A). Sonications were performed with 10 ms pulse length, 1 Hz pulse repetition frequency, for a duration of 10 min (FIG. 5B).

To assess the unique ultrasound pressure required for droplet vaporization in each rat, a 3 min pressure ramp was performed following the intravenous administration of nanodroplets. The ultrasound pressure was increased from 0.45 MPa with increments of 8 kPa, until the subharmonic emission exceeded 3.5 times the baseline value. This pressure was set as the vaporization threshold, and the following 10 min treatment sonication was fixed at this pressure. For ultrasound only groups, a fixed pressure of 1.1 MPa was used, based on the mean pressure required for vaporization.

### Measuring Blood Pressure in Awake Rats

Blood pressure and heart rate were measured noninvasively in awake rats using a tail-cuff system (CODA system, Kent Scientific, Torrington, CT, USA). Rats were placed inside a cylindrical holder, on a heated platform (CODA system), under a blanket, and allowed to acclimatize for 10 min before starting the measurement. 15 cycles were performed, and the mean systolic and diastolic blood pressure, and heart rate were recorded. Recordings which included tail movement or insufficient blood volume were automatically excluded by the CODA system software. Rats were habituated by performing the blood pressure measurement every day for 5 consecutive days prior to the first treatment.

For hypertensive rats, tail temperature was assessed by measuring the base of the tail at three time points with an infrared thermometer (TW2, Thermoworks, Salt Lake City, UT, USA), (1) immediately after placement in the holder, (2) after 10 min of acclimatization and (3) immediately after measurement. All rats were weighed each day.

### Assessment of Plasma Epinephrine and Norepinephrine

Venous blood was collected before and 24 hr after treatment to quantity epinephrine and norepinephrine plasma levels (hormones associated with metabolic function and blood pressure) in SHRs. 0.6 mL of blood was collected via the tail vein catheter and placed into an Eppendorf tube lined with heparin. The samples were stored on ice for 30 min before spinning down at 1000 G for 10 min. The plasma was separated and stored at −80C until analysis. Epinephrine and norepinephrine were assessed in triplicate with an ELISA (Abnova, KA1877,Taipei, Taiwan) as per the manufacturer specifications.

### Immunohistochemistry of Spontaneously Hypertensive Rats

Rats were intracardially perfused with ice-cold normal saline and 4% paraformaldehyde (PFA) in 0.1 M phosphate buffer 90-minutes after treatment. Brains were harvested, post-fixed in 4% PFA overnight, and then equilibrated in 30% sucrose in 0.1 M phosphate buffer. Samples were then embedded in OCT, cryosectioned horizontally at 40 μm-thick free-floating sections, and stored in cryoprotectant at −20° C.

One SHR from each group was used for the C-Fos expression analysis: naïve, FUS only (vIPAG), and PBND+FUS (vIPAG). Horizontal sections at 5.8 mm depth from bregma were selected for immunofluorescent staining, based on the sonication target coordinates. Sections were rinsed in 0.3% Triton X-100 in 0.1 M PBS and then treated in blocking 10% donkey serum solution for 1 hr at room temperature. Sections were incubated in primary antibodies, anti-cfos (ab190289, 1:1000, Abcam Inc., Cambridge, MA, USA) and anti-NeuN (ab 104224, 1:1000, Abcam Inc.), overnight at 4° C. Sections were then washed and incubated with fluorescently-labeled secondary antibodies, donkey Alexa Fluor® 488, anti-mouse (A21202, 1:1000, Invitrogen, Eugene, OR, USA) and donkey Alexa Fluor® 568, anti-rabbit (A10042, 1:1000, Invitrogen) diluted in blocking buffer, for 2 hrs at room temperature. Sections were washed with PBS and mounted onto X-tra glass slides (Leica Microsystems, Wetzlar, Germany) with aqueous mounting media with DAPI (Fluoroshield™ withDAPI, Sigma-Aldrich Corporation, St. Louis, MO, USA). Slides were stored at 4° C. in the dark until imaging.

Sections were imaged at 20× magnification (512×512-pixel field of view) for C-Fos quantification and NeuN colocalization analysis in a 1 mm² region of interest as previously described (Lunde and Glover, 2020). The Colocalization Image Creator plugin (ImageJ, National Institutes of Health, Bethesda, Maryland, USA) was used to create two custom binary images based on the raw data of the multichannel input images. Background signal intensity was reduced with global thresholding for each channel to generate binarized images. Binary images were Boolean AND combined, macro-transformed to remove holes in cells, and filtered to remove cells that were less than 5 μm in size. The first binarized image included the DAPI and C-Fos channels, and represented the total number of C-Fos labelled cells. The second binarized image included the DAPI, NeuN, and C-Fos channels, and represented the signal overlap across the three input channels, indicating colocalization between NeuN and C-Fos. Next, the Colocalization Object Counter plugin (ImageJ) was used to automatically count the number of C-Fos-positive cells, and the number of NeuN-positive cells colocalized with C-Fos. The total number of C-Fos-positive cells and the total number of C-Fos-NeuN colocalized cells detected in the sonicated and unsonicated PAG region are reported. The proportion of C-Fos positive cells that also show NeuN expression was also calculated.

To investigate treatment safety, red blood cell extravasation and cell apoptosis were assessed with hematoxylin and eosin (H&E) staining and terminal deoxynucleotidyl transferase dUTP nick end labeling (TUNEL) staining (DeadEnd™ Colorimetric TUNEL System, Promega, Madison, WI, USA). All rats were sacrificed 7 days following the final treatment and intracardially perfused with saline and buffered formalin. Brains were excised, immersed in 10% neutral buffered formalin for 24 h, then transferred into 70% ethanol for 48 h before being embedded in paraffin. 5 μm thick axial sections were taken at 500 μm separation and stained.

### Statistical Analysis

Blood pressure and heart rate data is presented as individual data points or bars showing mean +/standard deviation with error bars. Significance is evaluated using paired and unpaired t-tests, where the level of significance is set at p<0.05. Correlations are assessed with Pearson's coefficient (R²).

### Example 2: Results

### Habituation to Blood Pressure Measurements

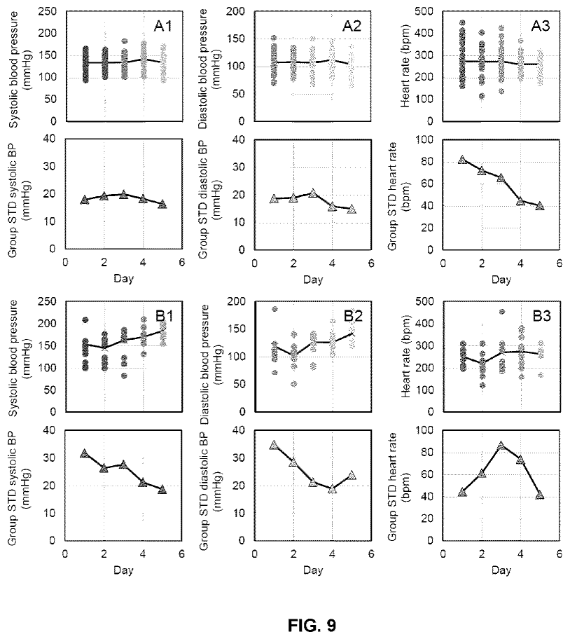

5 days of habituation was necessary for the rats to become tolerant of the non-invasive blood pressure measurement procedure (FIG. 9). After habituation, tail movement was significantly reduced during the measurement, and consistency of blood pressure and heart rate measurements was improved (FIG. 9). Following habituation, baseline systolic and diastolic blood pressure for normotensive rats was 133+/−16 mmHg and 103+/−14 mmHg respectively, with heart rate at 258 +/40 bpm. Baseline systolic and diastolic blood pressure for hypertensive rats (SHR) was 183 +/−18 mmHg and 142 +/−23 mmHg respectively, with heart rate at 263 +/−42 bpm.

### Transcranial Focused Ultrasound Temporarily Reduces Blood Pressure in Normotensive Rats

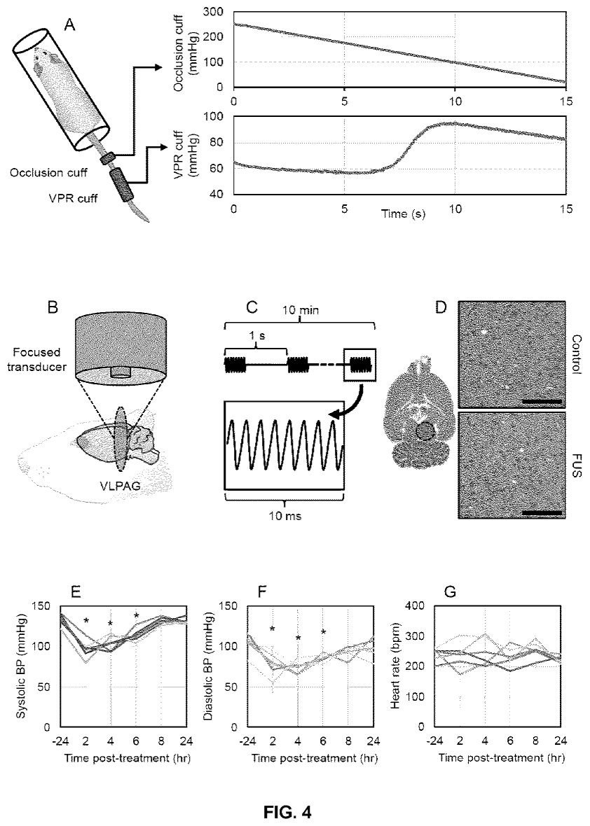

Following a single 10 min sonication of the vIPAG, 6 of 6 healthy Wistar rats showed a significant reduction in systolic (−37+/−7 mmHg) and diastolic (−28+/−9 mmHg) blood pressure, which remained significantly reduced from baseline at 6 hr after treatment (p<0.05, FIGS. 4E, 4F). Heart rate showed no significant change (FIG. 4G).

Transcranial Focused Ultrasound with Barbiturate Nanodroplets Sustains the Reduction in Blood Pressure in Normotensive Rats

General anesthesia (ISO only), and anesthesia combined with an injection of pentobarbital-loaded nanodroplets (ISO+PBND), showed no change in blood pressure when measured 2-48 hr following treatment (FIG. 5). Following a single sonication of the vIPAG region (FIG. 5A-5D), FUS-treated rats (ISO+FUS) had reduced systolic (−26+/−7 mmHg) and diastolic (−22+/−7 mmHg) blood pressure 2 hr after treatment. Sham nanodroplets (unloaded, not containing pentobarbital) combined with FUS (ISO+FUS+sham ND), also temporarily reduced blood pressure (−19+/−14 mmHg systolic, −15+/−18 mmHg diastolic). Combining FUS with pentobarbital-loaded nanodroplets (ISO+FUS+PBND) was found to sustain the effect, and blood pressure remained reduced for 24 hr following a single treatment (−25+/−5 mmHg systolic and −21+/−8 mmHg diastolic at 2 hr; −17+/−6 mmHg systolic and −18+/−10 mmHg diastolic at 24 hr).

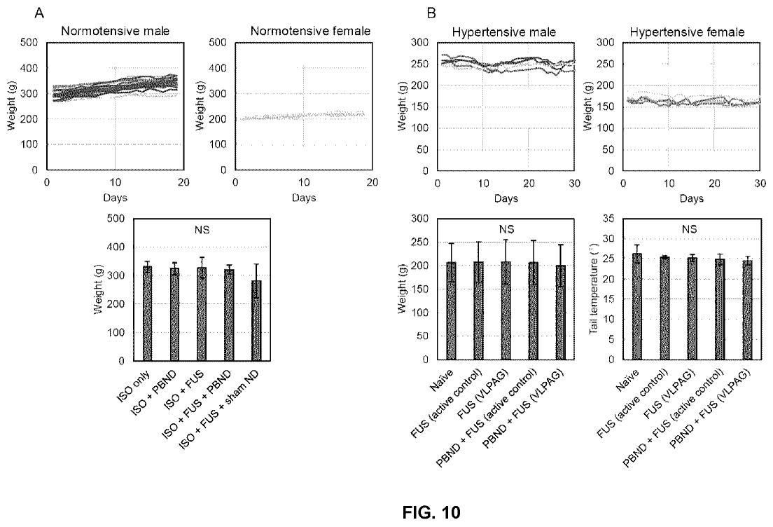

The following week, two treatments were performed on consecutive days (FIG. 5E). Again, ISO only and ISO+PBND, showed no change in blood pressure throughout 48 hr following either treatment. Following consecutive sonications of the vIPAG region, ISO+FUS reduced systolic and diastolic blood pressure 2 hr after both treatments (−28+/−5 mmHg systolic, −27+/−3 mmHg diastolic), as did sham nanodroplets and FUS (−29+/−13 mmHg systolic, −29+/−10 mmHg diastolic), and both treatment groups returned to baseline after 24 hr. Consecutive treatments of ISO+FUS+PBND sustained the effect: blood pressure remained reduced for 48 hr following two consecutive treatments (−26+/−7 mmHg systolic, −12+/−10 mmHg diastolic at 2 hr; −14 +/−10 mmHg systolic, −15+/−13 mmHg diastolic at 24 hr; −11+/−10 mmHg systolic, −9+/−11 mmHg diastolic at 48 hr). Changes in weight and heart rate were comparable to control rats (FIG. 10).

Transcranial Focused Ultrasound Reduces Blood Pressure in Hypertensive Rats and is Sustained with Barbiturate Nanodroplets

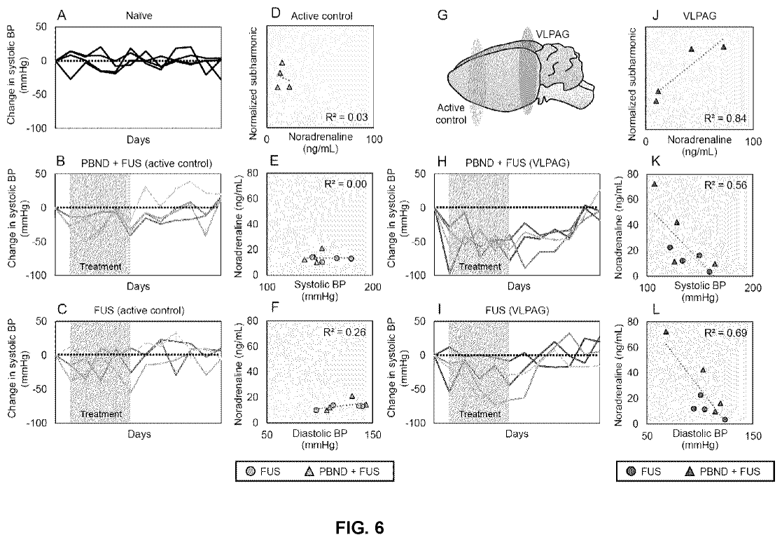

Male and female spontaneously hypertensive rats (SHR) were used to investigate FUS-mediated reduction in blood pressure, the efficacy of PBND, and the role of brain region in the treatment of hypertension. A crossover study was conducted, whereby 5 treatment groups of SHR received 5 consecutive treatments, spaced by 24 hr, followed by 6 days of monitoring, before being crossed over (FIG. 7A). FUS was applied to the left frontal cortex (a region not associated with regulating blood pressure, as an active control) (FUS (active control)) or applied to the vIPAG region (FUS (vIPAG)) (FIG. 6G).

FIG. 6 shows the change in systolic blood pressure for each SHR treated for 5 consecutive days, and monitored for 6 days following. Naïve SHR showed no change in blood pressure through the study (FIG. 6A). 3 of 4 hypertensive rats had reduced blood pressure following sonication of the vIPAG (FIG. 6I). FUS (vIPAG) showed a greater reduction in blood pressure (−43+/−29 mmHg systolic, −39+/−31 mmHg diastolic at 2 hr; −32+/−28 mmHg systolic, −38+/−34 mmHg diastolic at 24 hr) than FUS (active control) (−18+/−29 mmHg systolic, −11+/−27 mmHg diastolic at 2 hr; 1 +/−14 mmHg systolic, 6+/−13 mmHg diastolic at 24 hr) (FIGS. 6C, 6I). FUS (vIPAG) reduced blood pressure for 48 hr following 5 sonications and returned to baseline after 3 days.

4 of 4 hypertensive rats had reduced blood pressure following sonication of the vIPAG with PBND. PBND+FUS (vIPAG) showed the greatest and most consistent reduction in blood pressure (−57+/17 mmHg systolic, −48+/−17 mmHg diastolic at 2 hr), which remained significantly reduced for 4 (systolic) or 5 days (diastolic) following the last sonication (p<0.05). PBND+FUS (active control) showed a temporary reduction in blood pressure 2 hr after sonication (−37+/−16 mmHg systolic, −16+/−5 mmHg diastolic at 2 hr) and returned to baseline 24 hr after the final sonication. Animal weight and tail base temperature were comparable to naïve rats (FIG. 10).

At a sufficient ultrasound pressure, known as the vaporization threshold, nanodroplets transition from liquid to gas state, releasing the therapeutic and emitting a unique acoustic signal. Using linear regression analysis, the subharmonic and second harmonic emissions were found to correlate to the reduction in blood pressure (R²=0.88 and 0.75, Pearson's coefficient), whereas acoustic emissions detected from the active control region, or with FUS alone showed no correlation with change in blood pressure (R²<0.4, Pearson's coefficient).

### Plasma Hormone Content of Hypertensive Rats Correlates to Reduced Blood Pressure and Acoustic Response of Nanodroplets

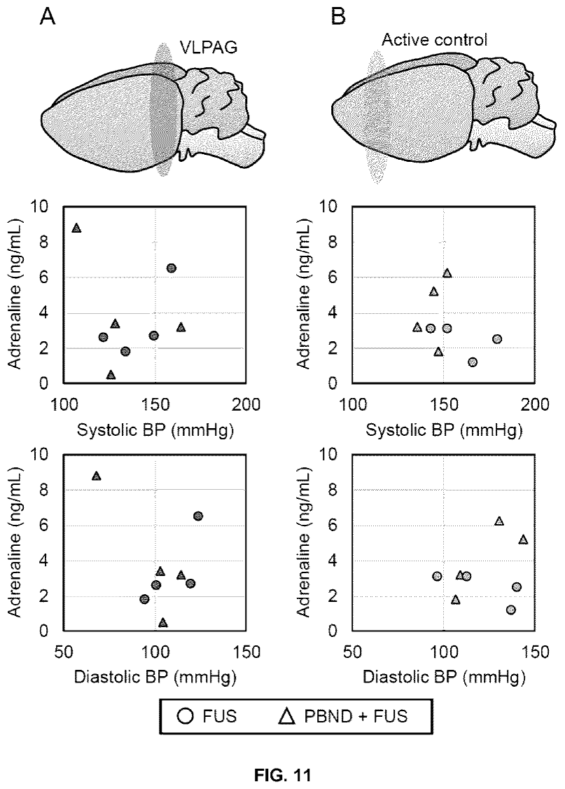

Adrenaline and noradrenaline plasma levels were quantified from plasma samples collected before, 24 hr after treatment and 1 week after treatment Mean noradrenaline level was higher for rats sonicated at the vIPAG (FIG. 6J-6L) compared to following sonication of the active control region (FIG. 6D-6F). 24 hr after sonication of the vIPAG region, noradrenaline levels showed a weak negative correlation with systolic (R²=0.56) (FIG. 7K) and diastolic (R²=0.69) (FIG. 6L) blood pressure, but no such relationship existed for the active control group (FIGS. 6E, 6F) or for the adrenaline levels (FIG. 11). Noradrenaline levels of hypertensive rats in PBND+FUS (vIPAG) group also mapped to subharmonic emissions (R²=0.84) (FIG. 6J). In contrast, hypertensive rats in PBND+FUS (active control) group showed no significant relationship between hormone levels and blood pressure or acoustic emissions (FIG. 6D-6F).

### Immunohistochemistry Assessment Maps Neuronal Stimulation of FUS

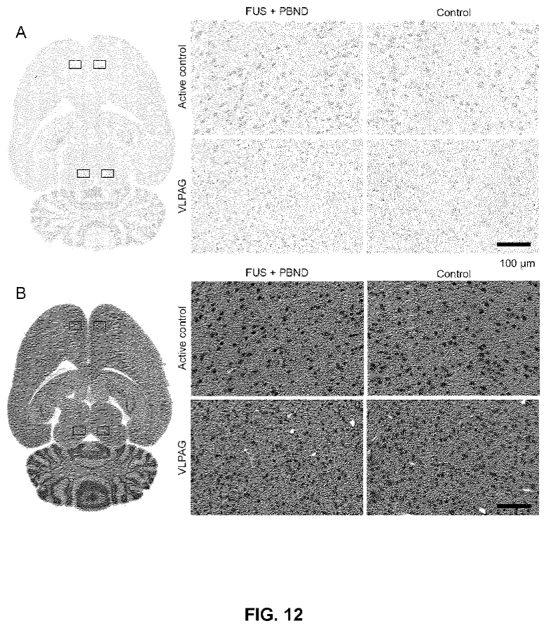

Three animals were perfused 90 min after treatment to assess C-Fos expression and NeuN antibodies (FIG. 8). FUS and FUS in combination with pentobarbital-loaded nanodroplets (PBND) showed a greater number of C-Fos positive cells in the sonicated region of the VLPAG compared to the contralateral side (FIG. 8A). 79% of C-Fos positive cells were also found to express NeuN following FUS only, and 61% of C-Fos positive cells expressed NeuN following PBND+FUS (FIG. 8G). H&E and TUNEL staining performed 7 days after the final treatment showed no signs of hemorrhage or edema and no increase in the number of apoptotic cells (FIG. 12).

### Example 3: Experimental Demonstration that Ultrasound-Mediated Reduction in Blood Pressure is Dose-Dependent

Many treatments for hypertension are ineffective for certain patient populations in cases such as drug-resistance and pregnancy. Previously, daily consecutive focused ultrasound (FUS) stimulation of the ventrolateral periaqueductal grey (vIPAG) in spontaneously hypertensive rats reduced blood pressure (BP) for up to 24 hr. Here, the effects of ultrasound pressure amplitude on the resulting reduction in blood pressure were investigated.

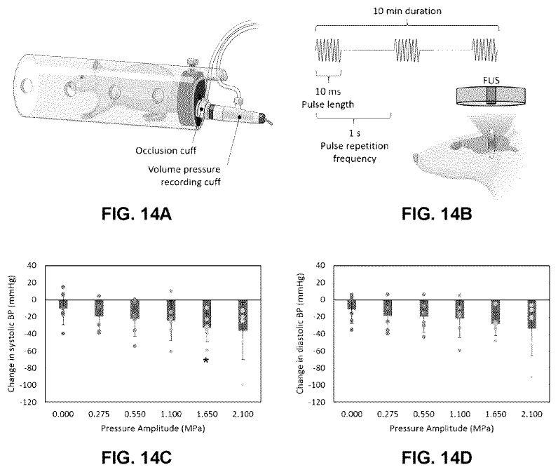

Normotensive Wistar rats (18 male, 18 female) were used to assess BP reduction following FUS stimulation. BP was measured using a non-invasive tail-cuff system (CODA, Kent Scientific) in awake, habituated rats (FIG. 14A). The vIPAG was targeted using a single element focused transducer (540 kHz, 34 mm element diameter, 3 mm beam width) with a stereotactic-guided positioning system (FUS Instruments). Sonications (10 min duration, 10 ms burst length, 1 Hz pulse repetition frequency) were performed in anesthetized rats (isoflurane and medical air), with six pressure amplitudes (0.000, 0.275, 0.550, 1.100, 1.650, and 2.100 MPa) (FIG. 14B). Immunohistochemistry was performed 90 mins following FUS stimulation to map neuronal firing and neuron subtype, and histology was performed 24 hr following FUS stimulation to assess safety.

A pressure-dependent reduction in BP was observed 2 hr post-FUS, reducing from the lowest pressure (0.275 MPa, −19.6±17.8 mmHg systolic, −19.3±17.6mmHg diastolic) to the highest (2.100 MPa, −36.6±33.4 mmHg systolic, −34.1±31.8 mmHg diastolic) (FIGS. 14C and 14D). At 24 hr post-FUS, BP returned to baseline for all ultrasound pressure amplitudes. There was a significant correlation between ultrasound pressure and reduction in systolic BP (p=0.02), with a slope of −11.1 mmHg/MPa. At the highest pressure, the change in systolic BP was linearly correlated with the baseline systolic BP (R²=0.65, p=0.05), suggesting that ultrasound-mediated BP reduction is most effective in subjects with high resting BP, as in the context of hypertension. Following exposure, no histological signs of hemorrhage, edema, or tissue damage were present. This data shows that ultrasound-mediated BP reduction is dose-dependent.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

### REFERENCES

Airan, R. D. et al. (20 17) ‘Noninvasive Targeted Transcranial Neuromodulation via Focused Ultrasound Gated Drug Release from Nanoemulsions’, Nano letters. 2017/01/17. American Chemical Society, 17(2), pp. 652-659. doi: 10.1021/acs.nanolett.6b03517.

Azizi, M. et al. (202 1) ‘Ultrasound renal denervation for hypertension resistant to a triple medication pill (RADIANCE-HTN TRIO): a randomised, multicentre, single-blind, sham-controlled trial’, The Lancet. Elsevier, 397(10293), pp. 2476-2486.

Basbaum, A. I. and Fields, H. L. (1984) ‘Endogenous pain control systems: brainstem spinal pathways and endorphin circuitry’, Annual review of neuroscience. Annual Reviews 4139 El Camino Way, PO Box 10139, Palo Alto, CA 94303-0139, USA, 7(1), pp. 309-338.

Behbehani, M. M. et al. (1990) ‘The effect of GABA and its antagonists on midbrain periaqueductal gray neurons in the rat’, Pain. Elsevier, 40(2), pp. 195-204.

Cui, M. et al. (1999) ‘Periaqueductal gray stimulation-induced inhibition of nociceptive dorsal horn neurons in rats is associated with the release of norepinephrine, serotonin, and amino acids’, Journal of Pharmacology and Experimental Therapeutics. ASPET, 289(2), pp. 868-876.

Davidson, B. et al. (2020) ‘Magnetic resonance-guided focused ultrasound capsulotomy for refractory obsessive compulsive disorder and major depressive disorder: clinical and imaging results from two phase I trials’, Molecular psychiatry. Nature Publishing Group, 25(9), pp. 1946-1957.

Elias, W. J. et al. (20 16) ‘A randomized trial of focused ultrasound thalamotomy for essential tremor’, New England Journal of Medicine. Mass Medical Soc, 375(8), pp. 730-739.

Esler, M. et al. (1984) ‘Plasma noradrenaline kinetics in humans’, Journal of the autonomic nervous system. Elsevier, 11(2), pp. 125-144.

Fenoy, A.J. and Simpson, R. (20 14) ‘Risks of common complications in deep brain stimulation surgery: management and avoidance’, Journal of Neurosurgery JNS. American Association of Neurological Surgeons, 120(1), pp. 132-139. doi: 10.3171/2013.10.JNS131225.

Fomenko, A. et al. (20 18) ‘Low-intensity ultrasound neuromodulation: An overview of mechanisms and emerging human applications’, Brain Stimulation, 11(6), pp. 1209-1217. doi: https://doi.org/10.1016/j.brs.2018.08.013.

FUSfoundation (2021) State of the field report. Available at: https://www.fusfoundation.org/images/pdf/FUSF_State_of_the_Field_2021_Final_Web.pdf.

Green, A. L. et al. (200 7) ‘Deep brain stimulation: a new treatment for hypertension?’, Journal of clinical neuroscience. Elsevier, 14(6), pp. 592-595.

Grimm, M. et al. (1980) ‘Norepinephrine clearance and pressor effect in normal and hypertensive man’, Klinische Wochenschrift. Springer, 58(21), pp. 1175-1181.

Hakimova, H. et al. (20 15) ‘Ultrasound stimulation inhibits recurrent seizures and improves behavioral outcome in an experimental model of mesial temporal lobe epilepsy’, Epilepsy & Behavior. Elsevier, 49, pp. 26-32.

Laurent, S. (20 17) ‘Antihypertensive drugs’, Pharmacological research. Elsevier, 124, pp. 116-125.

Lea-Banks, H. et al. (2020) ‘Localized anesthesia of a specific brain region using ultrasound-responsive barbiturate nanodroplets’, Theranostics, 10(6), pp. 2849-2858.

Lea-Banks, H. et al. (202 1) ‘Ultrasound-sensitive nanodroplets achieve targeted neuromodulation’, Journal of Controlled Release. Elsevier B. V., 332, pp. 30-39.doi: 10.1016/j.jconrel.2021.02.010.

Li, D. et al. (2020) ‘Low-intensity focused ultrasound stimulation treatment decreases blood pressure in spontaneously hypertensive rats’, IEEE Transactions on Biomedical Engineering. IEEE, 67(11), pp. 3048-3056.

Lunde, A. and Glover,. C. (2020) ‘A versatile toolbox for semi-automatic cell-by-cell object-based colocalization analysis’, Scientific reports. Nature Publishing Group, 10(1), pp. 1-26.

Martínez-Fernández, R. et al. (20 18) ‘Focused ultrasound subthalamotomy in patients with asymmetric Parkinson's disease: a pilot study’, The Lancet Neurology. Elsevier, 17(1), pp. 54-63.

Matsunaga, T. O. et al. (20 12) ‘Phase-change nanoparticles using highly volatile perfluorocarbons: toward a platform for extravascular ultrasound imaging’, Theranostics. 2012/12/23. Ivyspring International Publisher, 2(12), pp. 1185-1198. doi: 10.7150/thno.4846.

Meng, Y. et al. (202 1) ‘Focused ultrasound neuromodulation’, International Review of Neurobiology. Elsevier, 159, pp. 221-240.

Mooney, S. J. et al. (20 16) ‘Focused ultrasound-induced neurogenesis requires an increase in blood-brain barrier permeability’, PloS one. Public Library of Science San Francisco, CA USA, 11(7), p. e0159892.

Nelson, L. E. et al. (2002) ‘The sedative component of anesthesia is mediated by GA BAA receptors in an endogenous sleep pathway’, Nature neuroscience. Nature Publishing Group, 5(10), pp. 979-984.

Ozdas, M. S. et al. (2020) ‘Non-invasive molecularly-specific millimeter-resolution manipulation of brain circuits by ultrasound-mediated aggregation and uncaging of drug carriers’, Nature communications. Nature Publishing Group, 11(1), pp. 1-15.

Patel, N. K. et al. (20 11) ‘Deep brain stimulation relieves refractory hypertension’, Neurology. AAN Enterprises, 76(4), pp. 405-407.

Pereira, E. A. C. et al. (20 10) ‘Sustained reduction of hypertension by deep brain stimulation’, Journal of Clinical Neuroscience. Elsevier, 17(1), pp. 124-127.

Podymow, T. and August, P. (20 11) ‘Antihypertensive drugs in pregnancy’, in Seminars in nephrology. Elsevier, pp. 70-85.

Scarcelli, T. et al. (20 14) ‘Stimulation of hippocampal neurogenesis by transcranial focused ultrasound and microbubbles in adult mice’, Brain stimulation. Elsevier, 7(2), pp. 304-307.

Sharabi, S. et al. (20 1) ‘Non-thermal focused ultrasound induced reversible reduction of essential tremor in a rat model’, Brain stimulation. Elsevier, 12(1), pp. 1-8.

Sheeran, P. S. et al. (20 11) ‘Formulation and Acoustic Studies of a New Phase-Shift Agent for Diagnostic and Therapeutic Ultrasound’, Langmuir. American Chemical Society, 27(17), pp. 10412-10420. doi: 10.1021/la2013705.

Sheeran, P. S. et al. (20 1) ‘More Than Bubbles: Creating Phase-Shift Droplets from Commercially Available Ultrasound Contrast Agents’, Ultrasound in Medicine and Biology. Elsevier, 43(2), pp. 531-540. doi: 10.1016/j.ultrasmedbio.2016.09.003.

Stern, J. M. et al. (202 1) ‘Safety of focused ultrasound neuromodulation in humans with temporal lobe epilepsy’, Brain Stimulation. Elsevier.

Wang, J. B. et al. (20 18) ‘Noninvasive Ultrasonic Drug Uncaging Maps Whole-Brain Functional Networks’, Neuron. Cell Press, 100(3), pp. 728-738.e7. doi: 10.1016/J.NEURON.2018.10.042.

WHO (2022) World health statistics 2022: monitoring health for the SDGs, sustainable development goals. Available at: https://cdn.who.int/media/docs/default-source/gho-documents/world-health-statistic-reports/worldhealthstatistics_2022.pdf?sfvrsn=6fbb4d17_3.

Zhang, M., Li, B., Liu, Y., et al. (202 1) ‘Different Modes of Low-Frequency Focused Ultrasound-Mediated Attenuation of Epilepsy Based on the Topological Theory’, Micromachines. Multidisciplinary Digital Publishing Institute, 12(8), p. 1001.

Zhang, M., Li, B., Lv, X., et al. (202) ‘Low-Intensity Focused Ultrasound-Mediated Attenuation of Acute Seizure Activity Based on EEG Brain Functional Connectivity’, Brain Sciences. Multidisciplinary Digital Publishing Institute, 11(6), p. 711.

Zhang, T. et al. (2022) ‘Transcranial focused ultrasound stimulation of periaqueductal gray for analgesia’, IEEE Transactions on Biomedical Engineering. IEEE.

1-14. (canceled)

15. A method of treating hypertension using focused transcranial ultrasound, the method comprising:
after intravenous injection of nanodroplets to a subject, the nanodroplets comprising a perfluorocarbon core and a lipid shell, the nanodroplets being loaded with a lipophilic anesthetic agent:
transcranially delivering focused ultrasound to a region of the brain to vaporize the nanodroplets and release the lipophilic anesthetic agent, the region of the brain being selected from the group consisting of the periaqueductal gray region, the locus coeruleus, the thalamus, the hypothalamus, the neocortex and the cerebellum.

16. The method according to claim 15, wherein the focused ultrasound is delivered to the periaqueductal gray region.

17. The method according to claim 16, wherein the focused ultrasound is delivered to the ventrolateral periaqueductal gray (vIPAG).

18. The method according to claim 15, wherein the focused ultrasound is delivered to the hypothalamic arcuate nucleus (ARC) or the rostral ventrolateral medulla (rVLM).

19. The method according to claim 16, wherein the lipophilic anesthetic agent is a barbiturate.

20. The method according to claim 19, wherein the barbiturate is selected from the group consisting of amobarbital, alphenal, butabarbital, butalbital, butethal, pentobarbital, phenobarbital, secobarbital, and thiopental.

21. The method according to claim 16, wherein the lipophilic anesthetic agent is a GABA_A receptor agonist.

22. The method according to claim 16, wherein the lipophilic anesthetic agent is selected from the group consisting of benzocaine, bupivacaine (Marcaine), ketamine, levobupivacaine, lidocaine, prilocaine, procaine, propofol, ropivacaine, and tetracaine.

23. The method according to claim 16, wherein the lipophilic anesthetic agent has a molecular weight less than 400 Da and a partition coefficient logP of approximately 2.

24. The method according to claim 16, wherein the focused ultrasound is transcranially delivered by controlling ultrasound transducers of a transcranial headset to deliver focused ultrasound energy to the periaqueductal gray region.

25. The method according to claim 24, wherein the transcranial headset comprises a support structure supporting the ultrasound transducers, and wherein the support structure conforms to an anatomical contour of at least a portion of the head of the subject.

26. The method according to claim 25, wherein known positions and orientations of the ultrasound transducers relative to volumetric image data associated with the subject are employed to control the ultrasound transducers for delivering the focused ultrasound energy to the periaqueductal gray region of the subject when the transcranial headset is worn by the subject.

27. The method according to claim 24, further comprising:
processing signals received from the ultrasound transducers to detect acoustic emissions generated by vaporization of the nanodroplets in response to the focused ultrasound energy; and
employing the detected acoustic emissions as feedback to control delivery of the focused ultrasound energy according to an association between nanodroplet vaporization and delivery of an effective dose of the lipophilic anesthetic agent within the periaqueductal gray region.

28. The method according to claim 27, further comprising inferring a plasma noradrenaline content based on a correlation between plasma noradrenaline content and the detected acoustic emissions.

29. The method according to claim 27, wherein the signals received from the ultrasound transducers are employed to detect one or more of subharmonic acoustic emissions and harmonic acoustic emissions.

30. The method according to claim 24, wherein the ultrasound transducers are controlled to generate mechanical stress on tissue within the periaqueductal gray region by forming two or more closely spaced ultrasound pressure nodes, wherein neighboring nodes are in different acoustic phase to create a force between the ultrasound pressure nodes that stimulates or inhibits neuronal activity.

31. The method according to claim 24, wherein the ultrasound transducers are controlled to focus the focused ultrasound energy to the ventrolateral periaqueductal gray (vIPAG).

32. A system for treating hypertension using focused transcranial ultrasound, the system comprising:
a transcranial headset configured to be worn on the head of a subject, the transcranial headset comprising a support structure and a plurality of ultrasound transducers supported by the support structure; and
control and processing circuitry configured to perform operations comprising:
controlling the ultrasound transducers of the transcranial headset to deliver focused ultrasound energy to a periaqueductal gray region of a brain, the focused ultrasound energy being configured to cause vaporization of intravenously administered nanodroplets loaded with a lipophilic anesthetic agent to release the lipophilic anesthetic agent within the periaqueductal gray region for treatment of hypertension;
processing signals received from the ultrasound transducers to detect acoustic emissions generated by vaporization of the nanodroplets in response to the focused ultrasound energy; and
employing the detected acoustic emissions as feedback to control delivery of the focused ultrasound energy according to an association between nanodroplet vaporization and delivery of an effective dose of the lipophilic anesthetic agent within the periaqueductal gray region.

33. The system according to claim 32, wherein the support structure of the transcranial headset conforms to an anatomical contour of at least a portion of the head of a specific subject; and
wherein the system further comprises a storage medium, the storage medium comprising:
volumetric image data associated with the specific subject; and
transducer registration data enabling registration of the positions and orientations of the ultrasound transducers relative to the volumetric image data; and
wherein the control and processing circuitry is configured to employ the positions and orientations of the ultrasound transducers relative to the volumetric image data to control the ultrasound transducers for delivering the focused ultrasound energy to the periaqueductal gray region of the specific subject when the transcranial headset is worn by the specific subject.

34. The system according to claim 32, wherein the control and processing circuitry is further configured to infer plasma noradrenaline content based on a correlation between plasma noradrenaline content and the detected acoustic emissions.

35. The system according to claim 32 wherein the ultrasound transducers are controlled to generate mechanical stress on tissue within the periaqueductal gray region by forming two or more closely spaced ultrasound pressure nodes, wherein neighboring nodes are in different acoustic phase to create a force between the ultrasound pressure nodes that stimulates or inhibits neuronal activity.

36. The system according to claim 32 wherein the signals received from the ultrasound transducers are employed to detect one or more of subharmonic acoustic emissions and harmonic acoustic emissions.

37. The system according to claim 32 wherein the ultrasound transducers are controlled to focus the focused ultrasound energy to the ventrolateral periaqueductal gray (vIPAG).
